(19) United States
(12) Patent Application Publication
(10) Pub. No.: US 2022/0211659 A1
(43) Pub. Date: Jul. 7, 2022

(54) PHARMACEUTICAL COMPOSITION, METHODS FOR TREATING AND USES THEREOF

(71) Applicant: Boehringer Ingelheim International GmbH, Ingelheim am Rhein (DE)

(72) Inventors: Uli Christian BROEDL, Mainz am Rhein (DE); Afshin SALSALI, Princeton, NJ (US); Hans-Juergen WOERLE, Grandvaux VD (CH)

(21) Appl. No.: 17/696,075

(22) Filed: Mar. 16, 2022

Related U.S. Application Data
(63) Continuation of application No. 16/904,709, filed on Jun. 18, 2020, now abandoned.
Application No. 16/904,709 is a continuation of application No. 16/285,774, filed on Feb. 26, 2019, now abandoned.
Application No. 16/285,774 is a continuation of application No. 16/037,052, filed on Jul. 17, 2018, now abandoned.
Application No. 16/037,052 is a continuation of application No. 15/456,631, filed on Mar. 13, 2017, now abandoned.
(60) Provisional application No. 62/420,062, filed on Nov. 10, 2016.
(60) Provisional application No. 62/309,008, filed on Mar. 16, 2016.

(51) Int. Cl. A61K 31/351; A61K 31/7048; A61K 9/20; A61K 45/06

(52) U.S. Cl. CPC A61K 31/351; A61K 31/7048; A61K 9/2018; A61K 45/06; A61K 9/20

(57) ABSTRACT

The present invention relates to methods for preventing or treating acute or chronic heart failure and for reducing the risk of cardiovascular death, hospitalization for heart failure and other conditions in patients with preserved or reduced ejection fraction by administering empagliflozin to the patient.

### TECHNICAL FIELD OF THE INVENTION

The present invention relates to methods for treating chronic heart failure, for reducing the risk of cardiovascular death, for reducing the risk of hospitalization for heart failure, for reducing all-cause mortality, for reducing the risk of all-cause hospitalization, for reducing the risk of new onset of atrial fibrillation and for improving health-related quality of life and/or the functional capacity in a patient with chronic heart failure. The present invention also relates to methods for treating, preventing, protecting against, reducing the risk of or delaying the occurrence of acute heart failure, including acute decompensated heart failure. Furthermore the present invention relates to methods for improving the renal function and for treating or preventing certain renal conditions and diseases in a patient with chronic heart failure. The present invention further relates to empagliflozin for use in methods for treating and/or preventing certain diseases or disorder or reducing the risk of or delaying the occurrence of certain diseases or disorders in a patient with chronic heart failure.

### BACKGROUND OF THE INVENTION

Heart failure (HF) is a clinical syndrome caused by the inability of the heart to provide adequate blood supply or by sustaining adequate blood supply at the expense of elevated left ventricular (LV) filling pressure. Patients with Heart Failure (HF) face a poor diagnosis, and about 50% of patients die from HF within 5 years. About 66% of patients with HF are non-diabetic patients. Total prevalence of HF worldwide was 26 million in 2013. In the US, more than 1 million HF hospitalizations occur every year. There is a considerable unmet need in HF. The overall goal for the treatment of HF is to prevent hospitalization and mortality, control symptoms, and improve quality of life. There are two types of HF: HF with reduced (HFrEF) or preserved (HFpEF) Ejection Fraction, the latter representing 50% of total HF. Both HFrEF and HFpEF are associated with high morbidity and mortality. Current treatment options for HFrEF are mainly based on administration of beta-blockers, ACEi, ARBs, ARNi, MRAs and diuretics. Despite these options, outcomes remain suboptimal. There are at present no effective treatments indicated for HFpEF, with treatment focused on symptom management and on comorbidities.

Therefore, there is an unmet medical need for methods for treating chronic heart failure, in particular in patients with HFrEF or HFpEF, with a good efficacy, with regard to disease-modifying properties and with regard to reduction of risk of mortality or hospitalization while at the same time showing an good safety profile.

### SUMMARY OF THE INVENTION

The present invention relates to a method for treating, preventing, protecting against or delaying the occurrence of chronic heart failure in a patient in need thereof comprising administering empagliflozin to the patient.

The present invention also relates to a method for reducing the risk of cardiovascular death in a patient with chronic heart failure comprising administering empagliflozin to the patient.

In addition the present invention relates to a method for reducing the risk of hospitalization (first and recurrent) for heart failure in a patient with chronic heart failure comprising administering empagliflozin to the patient.

Furthermore the present invention relates to a method for reducing all-cause mortality in a patient with chronic heart failure comprising administering empagliflozin to the patient.

Furthermore the present invention relates to a method for reducing the risk of all-cause hospitalization in a patient with chronic heart failure comprising administering empagliflozin to the patient.

The present invention also relates to a method for reducing the risk of new onset of atrial fibrillation in a patient with chronic heart failure comprising administering empagliflozin to the patient.

The present invention also relates to a method for treating, preventing, protecting against, reducing the risk of or delaying the occurrence of acute heart failure in a patient in need thereof comprising administering empagliflozin to the patient.

The present invention also relates to a method for treating, preventing, protecting against, reducing the risk of or delaying the occurrence of acute decompensated heart failure (ADHF) in a patient with chronic heart failure comprising administering empagliflozin to the patient.

The present invention also relates to a method for preventing, slowing or reversing the progression to macroalbuminuria in a patient with chronic heart failure comprising administering empagliflozin to the patient.

The present invention also relates to a method for improving the renal function or for renal protection in a patient with chronic heart failure comprising administering empagliflozin to the patient.

The present invention also relates to a method for treating, preventing, protecting against, reducing the risk of, delaying the occurrence of and/or delaying the progression of chronic kidney disease in a patient with chronic heart failure comprising administering empagliflozin to the patient.

The present invention also relates to a method for improving the health related quality of life and/or the functional capacity in a patient with chronic heart failure comprising administering empagliflozin to the patient.

The present invention further provides for empagliflozin or a pharmaceutical composition comprising empagliflozin optionally in combination with one or more other therapeutic substances for use as a medicament in any one of the methods described herein.

The present invention further provides for empagliflozin or a pharmaceutical composition comprising empagliflozin optionally in combination with one or more other therapeutic substances for use in a method for treatment, prevention or risk reduction in any one of the diseases or conditions described herein.

The present invention further provides for empagliflozin or a pharmaceutical composition comprising empagliflozin optionally in combination with one or more other therapeutic substances for use in the manufacture of a medicament for use in any one of the methods described herein.

In one embodiment, the present invention provides a method of treatment comprising:
a) identifying a patient in need of treatment for chronic heart failure; and
b) administering empagliflozin to said patient.

In one embodiment, the present invention provides a method of treating chronic heart failure in a patient comprising:
a. determining the symptoms according to the NYHA classification of the patient;
b. identifying that the patient has chronic heart failure according to NYHA class I;
c. administering empagliflozin to the patient.

In one embodiment, the present invention provides a method of treating chronic heart failure in a patient comprising:
a. determining the symptoms according to the NYHA classification of the patient;
b. identifying that the patient has chronic heart failure according to NYHA class II;
c. administering empagliflozin to the patient.

In one embodiment, the present invention provides a method of treating chronic heart failure in a patient comprising:
a. determining the symptoms according to the NYHA classification of the patient;
b. identifying that the patient has chronic heart failure according to NYHA class III;
c. administering empagliflozin to the patient.

In one embodiment, the present invention provides a method of treating chronic heart failure in a patient comprising:
a. determining the symptoms according to the NYHA classification of the patient;
b. identifying that the patient has chronic heart failure according to NYHA class IV;
c. administering empagliflozin to the patient.

In one embodiment, the present invention provides a method of treating chronic heart failure in a patient comprising:
a. determining the ejection fraction of the patient;
b. identifying that the patient has an ejection fraction equal or smaller than 40%;
c. administering empagliflozin to the patient.

In one embodiment, the present invention provides a method of treating chronic heart failure in a patient comprising:
a. determining the symptoms according to the NYHA classification of the patient;
b. determining the ejection fraction of the patient;
c. identifying that the patient has chronic heart failure according to NYHA class I and has an ejection fraction equal or smaller than 40%;
d. administering empagliflozin to the patient.

In one embodiment, the present invention provides a method of treating chronic heart failure in a patient comprising:
a. determining the symptoms according to the NYHA classification of the patient;
b. determining the ejection fraction of the patient;
c. identifying that the patient has chronic heart failure according to NYHA class II, III or IV and has an ejection fraction equal or smaller than 40%;
d. administering empagliflozin to the patient.

In one embodiment, the present invention provides a method of treating chronic heart failure in a patient comprising:
a. determining the symptoms according to the NYHA classification of the patient;
b. determining the ejection fraction of the patient;
c. identifying that the patient has chronic heart failure according to NYHA class I and has an ejection fraction greater than 40%, in particular greater than 50%;
d. administering empagliflozin to the patient.

In one embodiment, the present invention provides a method of treating chronic heart failure in a patient comprising:
a. determining the symptoms according to the NYHA classification of the patient;
b. determining the ejection fraction of the patient;
c. identifying that the patient has chronic heart failure according to NYHA class II, III or IV and has an ejection fraction greater than 40%, in particular greater than 50%;
d. administering empagliflozin to the patient.

In one embodiment, the present invention provides a method of treating chronic heart failure in a patient comprising:
a. determining the symptoms according to the NYHA classification of the patient;
b. determining the ejection fraction of the patient;
c. determining the BNP or NT-proBNP value of the patient;
d. identifying that the patient has chronic heart failure according to NYHA class I and has an ejection fraction equal or smaller than 40%, in particular greater than 50%, and has an elevated BNP or NT-proBNP value;
e. administering empagliflozin to the patient.

In one embodiment, the present invention provides a method of treating chronic heart failure in a patient comprising:
a. determining the symptoms according to the NYHA classification of the patient;
b. determining the ejection fraction of the patient;
c. determining the BNP or NT-proBNP value of the patient;
d. identifying that the patient has chronic heart failure according to NYHA class II, III or IV and has an ejection fraction equal or smaller than 40%, in particular greater than 50%, and has an elevated BNP or NT-proBNP value;
e. administering empagliflozin to the patient.

According to this embodiment an elevated BNP or NT-proBNP value is particularly a BNP value equal to or greater than 150 pg/mL or a NT-proBNP value equal to or greater than 600 pg/mL. Furthermore according to this embodiment an elevated BNP or NT-proBNP value is particularly a BNP value equal to or greater than 100 pg/mL or a NT-proBNP value equal to or greater than 400 pg/mL if the patient was hospitalized for heart failure within the last 9 months.

In the methods according to the present invention empagliflozin is optionally administered in combination with one or more other therapeutic substances to the patient.

Further aspects of the present invention become apparent to the one skilled in the art by the description hereinbefore and in the following and by the examples.

Definitions

The term “active ingredient” of a pharmaceutical composition according to the present invention means the SGLT2 inhibitor empagliflozin according to the present invention. An “active ingredient” is also sometimes referred to herein as an “active substance”.

The term “body mass index” or “BMI” of a human patient is defined as the weight in kilograms divided by the square of the height in meters, such that BMI has units of kg/m².

The term “overweight” is defined as the condition wherein the individual has a BMI greater than or 25 kg/m² and less than 30 kg/m². The terms “overweight” and “pre-obese” are used interchangeably.

The terms “obesity” or “being obese” and the like are defined as the condition wherein the individual has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the term “class III obesity” is the condition wherein the BMI is equal to or greater than 40 kg/m².

The indication obesity includes in particular exogenic obesity, hyperinsulinaemic obesity, hyperplasmic obesity, hyperphyseal adiposity, hypoplasmic obesity, hypothyroid obesity, hypothalamic obesity, symptomatic obesity, infantile obesity, upper body obesity, alimentary obesity, hypogonadal obesity, central obesity, visceral obesity, abdominal obesity.

The term “visceral obesity” is defined as the condition wherein a waist-to-hip ratio of greater than or equal to 1.0 in men and 0.8 in women is measured. It defines the risk for insulin resistance and the development of pre-diabetes.

The term “abdominal obesity” is usually defined as the condition wherein the waist circumference is >40 inches or 102 cm in men, and is >35 inches or 94 cm in women. With regard to a Japanese ethnicity or Japanese patients abdominal obesity may be defined as waist circumference≥85 cm in men and ≥90 cm in women (see e.g. investigating committee for the diagnosis of metabolic syndrome in Japan).

The term “euglycemia” is defined as the condition in which a subject has a fasting blood glucose concentration within the normal range, greater than 70 mg/dL (3.89 mmol/L) and less than 100 mg/dL (5.6 mmol/L). The word “fasting” has the usual meaning as a medical term.

The term “hyperglycemia” is defined as the condition in which a subject has a fasting blood glucose concentration above the normal range, greater than 100 mg/dL (5.6 mmol/L). The word “fasting” has the usual meaning as a medical term.

The term “hypoglycemia” is defined as the condition in which a subject has a blood glucose concentration below the normal range, in particular below 70 mg/dL (3.89 mmol/L).

The term “postprandial hyperglycemia” is defined as the condition in which a subject has a 2 hour postprandial blood glucose or serum glucose concentration greater than 200 mg/dL (11.11 mmol/L).

The term “impaired fasting blood glucose” or “IFG” is defined as the condition in which a subject has a fasting blood glucose concentration or fasting serum glucose concentration in a range from 100 to 125 mg/dl (i.e. from 5.6 to 6.9 mmol/l), in particular greater than 110 mg/dL and less than 126 mg/dl (7.00 mmol/L). A subject with “normal fasting glucose” has a fasting glucose concentration smaller than 100 mg/dl, i.e. smaller than 5.6 mmol/l.

The term “impaired glucose tolerance” or “IGT” is defined as the condition in which a subject has a 2 hour postprandial blood glucose or serum glucose concentration greater than 140 mg/dl (7.78 mmol/L) and less than 200 mg/dL (11.11 mmol/L). The abnormal glucose tolerance, i.e. the 2 hour postprandial blood glucose or serum glucose concentration can be measured as the blood sugar level in mg of glucose per dL of plasma 2 hours after taking 75 g of glucose after a fast. A subject with “normal glucose tolerance” has a 2 hour postprandial blood glucose or serum glucose concentration smaller than 140 mg/dl (7.78 mmol/L).

The term “hyperinsulinemia” is defined as the condition in which a subject with insulin resistance, with or without euglycemia, has fasting or postprandial serum or plasma insulin concentration elevated above that of normal, lean individuals without insulin resistance, having a waist-to-hip ratio<1.0 (for men) or <0.8 (for women).

The terms “insulin-sensitizing”, “insulin resistance-improving” or “insulin resistance-lowering” are synonymous and used interchangeably.

The term “insulin resistance” is defined as a state in which circulating insulin levels in excess of the normal response to a glucose load are required to maintain the euglycemic state (Ford E S, et al. JAMA. (2002) 287:356-9). A method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. With this method, it is not possible to distinguish between hepatic and peripheral insulin resistance.

Furthermore, insulin resistance, the response of a patient with insulin resistance to therapy, insulin sensitivity and hyperinsulinemia may be quantified by assessing the “homeostasis model assessment to insulin resistance (HOMA-IR)” score, a reliable indicator of insulin resistance (Katsuki A, et al. Diabetes Care 2001; 24: 362-5). Further reference is made to methods for the determination of the HOMA-index for insulin sensitivity (Matthews et al., Diabetologia 1985, 28: 412-19), of the ratio of intact proinsulin to insulin (Forst et al., Diabetes 2003, 52(Suppl.1): A459) and to an euglycemic clamp study. In addition, plasma adiponectin levels can be monitored as a potential surrogate of insulin sensitivity. The estimate of insulin resistance by the homeostasis assessment model (HOMA)-IR score is calculated with the formula (Galvin P, et al. Diabet Med 1992;9:921-8):

HOMA-IR=[fasting serum insulin (μU/mL)]×[fasting plasma glucose(mmol/L)/22.5]

Insulin resistance can be confirmed in these individuals by calculating the HOMA-IR score. For the purpose of this invention, insulin resistance is defined as the clinical condition in which an individual has a HOMA-IR score>4.0 or a HOMA-IR score above the upper limit of normal as defined for the laboratory performing the glucose and insulin assays.

As a rule, other parameters are used in everyday clinical practice to assess insulin resistance. Preferably, the patient's triglyceride concentration is used, for example, as increased triglyceride levels correlate significantly with the presence of insulin resistance.

Individuals likely to have insulin resistance are those who have two or more of the following attributes: 1) overweight or obese, 2) high blood pressure, 3) hyperlipidemia, 4) one or more 1^{st} degree relative with a diagnosis of IGT or IFG or type 2 diabetes.

Patients with a predisposition for the development of IGT or IFG or type 2 diabetes are those having euglycemia with hyperinsulinemia and are by definition, insulin resistant. A typical patient with insulin resistance is usually overweight or obese. If insulin resistance can be detected, this is a particularly strong indication of the presence of pre-diabetes. Thus, it may be that in order to maintain glucose homoeostasis a person needs 2-3 times as much insulin as a healthy person, without this resulting in any clinical symptoms.

“Pre-diabetes” is a general term that refers to an intermediate stage between normal glucose tolerance (NGT) and overt type 2 diabetes mellitus (T2DM), also referred to as intermediate hyperglycaemia. Therefore in one aspect of the present invention “pre-diabetes” is diagnosed in an individual if HbA1c is more or equal to 5.7% and less than 6.5%. According to another aspect of this invention “pre-diabetes” represents 3 groups of individuals, those with impaired glucose tolerance (IGT) alone, those with impaired fasting glucose (IFG) alone or those with both IGT and IFG. IGT and IFG usually have distinct pathophysiologic etiologies, however also a mixed condition with features of both can exist in patients. Therefore in another aspect of the present invention a patient being diagnosed of having “pre-diabetes” is an individual with diagnosed IGT or diagnosed IFG or diagnosed with both IGT and IFG. Following the definition according to the American Diabetes Association (ADA) and in the context an aspect of the present invention a patient being diagnosed of having “pre-diabetes” is an individual with:

a) a fasting plasma glucose (FPG) concentration<100 mg/dL [1 mg/dL=0.05555 mmol/L]and a 2-hour plasma glucose (PG) concentration, measured by a 75-g oral glucose tolerance test (OGTT), ranging between ≥140 mg/dL and <200 mg/dL (i.e., IGT); or

b) a fasting plasma glucose (FPG) concentration between ≥100 mg/dL and <126 mg/dL and a 2-hour plasma glucose (PG) concentration, measured by a 75-g oral glucose tolerance test (OGTT) of <140 mg/dL (i.e., IFG); or

c) a fasting plasma glucose (FPG) concentration between ≥100 mg/dL and <126 mg/dL and a 2-hour plasma glucose (PG) concentration, measured by a 75-g oral glucose tolerance test (OGTT), ranging between ≥140 mg/dL and <200 mg/dL (i.e., both IGT and IFG).

Patients with “pre-diabetes” are individuals being pre-disposed to the development of type 2 diabetes. Pre-diabetes extends the definition of IGT to include individuals with a fasting blood glucose within the high normal range≥100 mg/dL (J. B. Meigs, et al. Diabetes 2003; 52:1475-1484). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749).

The methods to investigate the function of pancreatic beta-cells are similar to the above methods with regard to insulin sensitivity, hyperinsulinemia or insulin resistance: An improvement of beta-cell function can be measured for example by determining a HOMA-index (homeostasis model assessment) for beta-cell function, HOMA-B, (Matthews et al., Diabetologia 1985, 28: 412-19), the ratio of intact proinsulin to insulin (Forst et al., Diabetes 2003, 52(Suppl.1): A459), first and second phase insulin secretion after an oral glucose tolerance test or a meal tolerance test (Stumvoll et al., Diabetes care 2000, 23: 295-301), the insulin/C-peptide secretion after an oral glucose tolerance test or a meal tolerance test, or by employing a hyperglycemic clamp study and/or minimal modeling after a frequently sampled intravenous glucose tolerance test (Stumvoll et al., Eur J Clin Invest 2001, 31: 380-81).

The term “type 1 diabetes” is defined as the condition in which a subject has, in the presence of autoimmunity towards the pancreatic beta-cell or insulin, a fasting blood glucose or serum glucose concentration greater than 125 mg/dL (6.94 mmol/L). If a glucose tolerance test is carried out, the blood sugar level of a diabetic will be in excess of 200 mg of glucose per dL (11.1 mmol/l) of plasma 2 hours after 75 g of glucose have been taken on an empty stomach, in the presence of autoimmunity towards the pancreatic beta cell or insulin. In a glucose tolerance test 75 g of glucose are administered orally to the patient being tested after 10-12 hours of fasting and the blood sugar level is recorded immediately before taking the glucose and 1 and 2 hours after taking it. The presence of autoimmunity towards the pancreatic beta-cell may be observed by detection of circulating islet cell autoantibodies [“type 1A diabetes mellitus”], i.e., at least one of: GAD65 [glutamic acid decarboxylase-65], ICA [islet-cell cytoplasm], IA-2 [intracytoplasmatic domain of the tyrosine phosphatase-like protein IA-2], ZnT8 [zinc-transporter-8] or anti-insulin; or other signs of autoimmunity without the presence of typical circulating autoantibodies [type 1B diabetes], i.e. as detected through pancreatic biopsy or imaging). Typically a genetic predisposition is present (e.g. HLA, INS VNTR and PTPN22), but this is not always the case.

The term “type 2 diabetes mellitus” or “T2DM” is defined as the condition in which a subject has a fasting blood glucose or serum glucose concentration greater than 125 mg/dL (6.94 mmol/L). The measurement of blood glucose values is a standard procedure in routine medical analysis. If a glucose tolerance test is carried out, the blood sugar level of a diabetic will be in excess of 200 mg of glucose per dL (11.1 mmol/l) of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject, the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg, this is regarded as abnormal glucose tolerance.

The term “late stage type 2 diabetes mellitus” includes patients with a secondary drug failure, indication for insulin therapy and progression to micro- and macrovascular complications e.g. diabetic nephropathy, or coronary heart disease (CHD).

The term “LADA” (“latent autoimmune diabetes of adults”) refers to patients that have a clinical diagnosis of type 2 diabetes, but who are being detected to have autoimmunity towards the pancreatic beta cell. Latent autoimmune diabetes of adults (LADA) is also known as slowly progressive type 1 diabetes mellitus (T1DM), “mild” T1 DM, non-insulin dependent type 1 DM, type 1½ DM, double diabetes or antibody positive type 2 DM (T2DM). LADA is often not clearly defined and, opposed to T1 DM, seldom or never presents with significant weight loss and ketoacidosis due to rapidly progressive p-cell failure.

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example, metformin on its own achieves an average improvement in the HbA1c value in the diabetic of the order of 1.0-1.5%. This reduction of the HbA1C value is not sufficient in all diabetics to achieve the desired target range of <7% or <6.5% and preferably <6% HbA1c.

The term “insufficient glycemic control” or “inadequate glycemic control” in the scope of the present invention means a condition wherein patients show HbA1c values above 6.5%, in particular above 7.0%, even more preferably above 7.5%, especially above 8%.

The “metabolic syndrome”, also called “syndrome X” (when used in the context of a metabolic disorder), also called the “dysmetabolic syndrome” is a syndrome complex with the cardinal feature being insulin resistance (Laaksonen D E, et al. Am J Epidemiol 2002; 156:1070-7). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285:2486-2497), diagnosis of the metabolic syndrome is made when three or more of the following risk factors are present:
1. Abdominal obesity, defined as waist circumference >40 inches or 102 cm in men, and >35 inches or 94 cm in women; or with regard to a Japanese ethnicity or Japanese patients defined as waist circumference≥85 cm in men and ≥90 cm in women;
2. Triglycerides: 150 mg/dL
3. HDL-cholesterol <40 mg/dL in men
4. Blood pressure 130/85 mm Hg (SBP 130 or DBP 85)
5. Fasting blood glucose 100 mg/dL

The NCEP definitions have been validated (Laaksonen D E, et al. Am J Epidemiol. (2002) 156:1070-7). Triglycerides and HDL cholesterol in the blood can also be determined by standard methods in medical analysis and are described for example in Thomas L (Editor): “Labor and Diagnose”, TH-Books Verlagsgesellschaft mbH, Frankfurt/Main, 2000.

According to a commonly used definition, hypertension is diagnosed if the systolic blood pressure (SBP) exceeds a value of 140 mm Hg and diastolic blood pressure (DBP) exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg.

The term “empagliflozin” refers to the SGLT2 inhibitor 1-chloro-4-(β-D-glucopyranos-1-yl)-2-[4-((S)-tetrahydrofuran-3-yloxy)-benzyl]-benzene of the formula

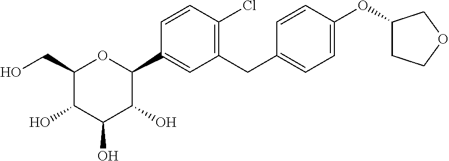 [SMILES: OC[C@H]1O[C@@H](c2ccc(Cl)c(Cc3ccc(O[C@H]4CCOC4)cc3)c2)[C@H](O)[C@@H](O)[C@@H]1O]

as described for example in WO 2005/092877. Methods of synthesis are described in the literature, for example WO 06/120208 and WO 2011/039108. According to this invention, it is to be understood that the definition of empagliflozin also comprises its hydrates, solvates and polymorphic forms thereof, and prodrugs thereof. An advantageous crystalline form of empagliflozin is described in WO 2006/117359 and WO 2011/039107 which hereby are incorporated herein in their entirety. This crystalline form possesses good solubility properties which enables a good bioavailability of the SGLT2 inhibitor. Furthermore, the crystalline form is physico-chemically stable and thus provides a good shelf-life stability of the pharmaceutical composition. Preferred pharmaceutical compositions, such as solid formulations for oral administration, for example tablets, are described in WO 2010/092126, which hereby is incorporated herein in its entirety.

The terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present invention may be used for instance as therapeutic treatment over a period of time as well as for chronic therapy.

The terms “prophylactically treating”, “preventivally treating” and “preventing” are used interchangeably and comprise a treatment of patients at risk to develop a condition mentioned hereinbefore, thus reducing said risk.

The term “tablet” comprises tablets without a coating and tablets with one or more coatings. Furthermore the “term” tablet comprises tablets having one, two, three or even more layers and press-coated tablets, wherein each of the beforementioned types of tablets may be without or with one or more coatings. The term “tablet” also comprises mini, melt, chewable, effervescent and orally disintegrating tablets.

The terms “pharmacopoe” and “pharmacopoeias” refer to standard pharmacopoeias such as the “USP 31-NF 26 through Second Supplement” (United States Pharmacopeial Convention) or the “European Pharmacopoeia 6.3” (European Directorate for the Quality of Medicines and Health Care, 2000-2009).

The term “chronic heart failure” or “CHF” is a synonym of congestive heart failure (CCF). The extent of heart failure may be classified according to the New York Heart Association (NYHA) Functional Classification and encompasses the NYHA classes I, II, III and IV. Chronic heart failure may be distinguished according to the ability of the left ventricle to contract is affected (heart failure with reduced ejection fraction) or the heart's abiility to relax is affected (heart failure with preserved ejection fraction).

The term “HFpEF” refers to heart failure with preserved ejection fraction. HFpEF is sometimes also referred to as “Diastolic Heart Failure”.

The term “HFrEF” refers to heart failure with reduced ejection fraction. HFrEF is sometimes also referred to as “Systolic Heart Failure”.

The term “LVEF” refers to the left ventricular ejection fraction.

The ejection fraction may be obtained by echocardiography, radionuclide ventriculography and angiography, preferably by echocardiography.

The term “BNP” refers to the brain natriuretic peptide, also called B-type natriuretic peptide. BNP is used for screening and diagnosis for chronic heart failure. The BNP value is determined in the blood plasma or serum.

The term “NT-proBNP” refers to the N-terminal of the prohormone brain natriuretic peptide. NT-proBNP is used for screening and diagnosis for chronic heart failure. The NT-proBNP value is determined in the blood plasma or serum.

The term “albuminuria” is defined as a condition wherein more than the normal amount of albumin is present in the urine. Albuminuria can be determined by the albumin excretion rate (AER) and/or the albumin-to-creatine ratio (ACR) in the urine (also refered to as UACR). Albuminuria categories in CKD are defined as follows:

| | AER (mg/ | ACR (approximate equivalent) | | |
|---|---|---|---|---|
| Category | 24 hours) | (mg/mmol) | (mg/g) | Terms |
| A1 | <30 | <3 | <30 | Normal to mildly |
| | | | | increased |
| A2 | 30-300 | 3-30 | 30-300 | Moderately |
| | | | | increased |
| A3 | >300 | >30 | >300 | Severely increased |

Category A1 reflects no albuminuria, category A2 reflects microalbuminuria, category A3 reflects macroalbuminuria. The progression of category A1 usually leads to microalbuminuria (A2) but may also directly result in macroalbuminuria (A3). Progression of microalbuminuria (A2) results in macroalbuminuria (A3).

The term “eGFR” refers to the estimated glomerular filtration rate (GFR). The GFR describes the flow rate of filtered fluid through the kidney. The estimated GFR may be calculated based on serum creatinine values e.g. using the Chronic Kidney Disease Epidemiology Collaboration (CKD-EPI) equation, the Cockcroft-Gault formula or the Modification of Diet in Renal Disease (MDRD) formula, which are all known in the art.

According to an aspect of this invention the estimated glomerular filtration rate (eGFR) is derived from serum creatinine values, age sex and race based on the CKD-EPI equation:

GFR=141×min (S_{cr}/κ, 1)^{α}×max(S_{cr}/κ, 1)^{-1.209}×0.993^{Age}×1.018 [if female]×1.159 [if black]

where:

Scr is serum creatinine in mg/dL,

κ is 0.7 for females and 0.9 for males,

α is −0.329 for females and −0.411 for males,

min indicates the minimum of S_{cr}/κ or 1, and

max indicates the maximum of S_{cr}/κ or 1.

For the purpose of the present invention, the degree of renal impairment in a patient is defined by the following estimated glomerular filtration rate (eGFR):

Normal renal function (CKD stage 1): eGFR≥90 mL/min/1.73 m²

Mild renal impairment (CKD stage 2): eGFR≥60 to <90 mL/min/1.73 m²

Moderate renal impairment (CKD stage 3): eGFR≥30 to <60 mL/min/1.73 m²

Severe renal impairment (CKD stage 4): eGFR≥15 to <30 mL/min/1.73 m²

Kidney failure (CKD stage 5): eGFR<15 mL/min/1.73 m²

According to the present invention moderate renal impairment can be further divided into two sub-stages:

Moderate A renal impairment (CKD 3A): eGFR≥45 to <60 mL/min/1.73 m²

Moderate B renal impairment (CKD 3B): eGFR≥30 to <45 mL/min/1.73 m²

The term “KCCQ” refers to Kansas City Cardiomyopathy Questionnaire. The health related quality of life may be measured according to KCCQ or KCCQ-12. KCCQ-12 is a validated short version of the original 23-item KCCQ (Kansas City Cardiomyopathy Questionnaire). This self-administered questionnaire is designed to evaluate physical limitations, symptoms (frequency, severity, and changes over time), social limitations, self-efficacy, and quality of life in patients with HF.

The term “MLHFQ” refers to Minnesota Living With Heart Failure Questionnaire. The quality of life, including for example its physical, emotional, social and mental dimensions, may be measured according to MLHFQ.

### DETAILED DESCRIPTION OF THE INVENTION

Beyond an improvement of glycemic control and weight loss due to an increase in urinary glucose excretion, empagliflozin shows a diuretic effect, reduced arterial stiffness and direct vascular effects (Cherney et al., Cardiovasc Diabetol. 2014; 13:28; Cherney et al., Circulation. 2014; 129:587-597). In the EMPA-REG OUTCOME™ study it was demonstrated that empagliflozin reduced the risk of cardiovascular death, hospitalization for heart failure and overall mortality in patients with type 2 diabetes mellitus and high cardiovascular risk (Zinman et al., N Engl J Med. 2015; 373:2117-2128). It was observed that treatment with empagliflozin leads to blood pressure reductions without clinically relevant changes of the heart rate, thus improving rate pressure product (RPP), a surrogate marker of cardiac oxygen demand. Furthermore empagliflozin was found of not being associated with clinically relevant reflex-mediated sympathetic activation in contrast to increases observed with diuretics. It may be assumed that altered glucose and sodium gradients within the kidney may generate a sympathoinhibitory afferent renal nerve signal. The lack of sympathetic activation may contribute to a beneficial cardiovascular and renal profile of empagliflozin (cardiorenal axis). Based on clinical and non-clinical studies including mechanistic considerations, such as the effect of empagliflozin on human autonomic cardiovascular regulation, the use of empagliflozin in the treatment and prevention of certain diseases and conditions, in particular in chronic heart failure, acute heart failure and chronic kidney disease, is described hereinbefore and hereinafter.

The present invention relates to a method for treating chronic heart failure in a patient in need thereof comprising administering empagliflozin to the patient. The present invention also relates to a method for reducing the risk of cardiovascular death in a patient with chronic heart failure comprising administering empagliflozin to the patient. The present invention further relates to a method for reducing the risk of hospitalization for heart failure in a patient with chronic heart failure comprising administering empagliflozin to the patient. The present invention also relates to a method for reducing the risk of cardiovascular death and hospitalization for heart failure in a patient with chronic heart failure comprising administering empagliflozin to the patient. According to an embodiment of this invention the risk of hospitalization for heart failure is the risk of first hospitalization for heart failure. According to another embodiment of this invention the risk of hospitalization for heart failure is the risk of recurrent hospitalization for heart failure. The present invention further relates to a method for reducing all-cause mortality in a patient with chronic heart failure comprising administering empagliflozin to the patient. Furthermore the present invention relates to a method for reducing the risk of all-cause hospitalization in a patient with chronic heart failure comprising administering empagliflozin to the patient. According to an embodiment of this invention the risk of all-cause hospitalization is the risk of first all-cause hospitalization. According to another embodiment of this invention the risk of all-cause hospitalization is the risk of recurrent all-cause hospitalization. The present invention also relates to a method for reducing the risk of new onset of atrial fibrillation in a patient with chronic heart failure comprising administering empagliflozin to the patient.

The present invention also relates to a method for preventing, protecting against or delaying the occurrence of chronic heart failure in a patient in need thereof comprising administering empagliflozin to the patient. According to an embodiment of this invention a method for preventing a worsening of chronic heart failure in a patient with chronic heart failure of NYHA class Ito chronic heart failure of NYHA class II, III or IV is provided.

The present invention also relates to a method for treating, preventing, protecting against or delaying the occurrence of acute heart failure in a patient in need thereof comprising administering empagliflozin to the patient, in particular wherein the patient is a patient with chronic heart failure.

The present invention also relates to a method for treating, preventing, protecting against, reducing the risk of or delaying the occurrence of acute decompensated heart failure (ADHF) in a patient with chronic heart failure in need thereof comprising administering empagliflozin to the patient.

In the methods according to the present invention the risk of a certain event, disease or disorder is reduced when compared to a patient administered with a placebo on standard of care background medication. In one embodiment, the risk is reduced by 15% or more. In one embodiment, the risk is reduced by 16% or more, by 17% or more, by 18% or more, by 19% or more, by 20% or more, by 25% or more or by 30% or more.

According to one embodiment of this invention the patient is a patient with chronic heart failure according to NYHA class II, III or IV.

According to an aspect of this embodiment of this invention the patient is a patient with chronic heart failure according to NYHA class II or III.

According to another embodiment of this invention the patient is a patient with chronic heart failure according to NYHA class I.

According to one embodiment of this invention the patient is a patient with chronic heart failure and preserved ejection fraction (HFpEF). For example the patient with preserved ejection fraction shows a LVEF greater than 40% or even greater than 50%. According to a variant of this embodiment the patient with chronic heart failure and preserved ejection fraction (HFpEF) shows a LVEF equal to or greater than 50%. According to another variant of this embodiment the patient shows a LVEF in a range from 40% to 49%, also called chronic heart failure with mid-range reduced ejection fraction (HFmrEF).

According to another embodiment of this invention the patient is a patient with chronic heart failure and reduced ejection fraction (HFrEF). For example the patient with reduced ejection fraction shows a LVEF of smaller or equal than 40%, in particular smaller than 40%.

Therefore according to an embodiment of this invention the invention provides a method for treating chronic heart failure with preserved ejection fraction (HFpEF) in a patient in need thereof comprising administering empagliflozin to the patient, for example in a patient with chronic heart failure according to NYHA class I, II, III or IV. According to an aspect of this embodiment the extent of chronic heart failure in a patient with chronic heart failure according to NYHA class II, III or IV is improved according to the NYHA classification. The patient according to this embodiment is for example a non-diabetic patient, a patient with pre-diabetes or a patient with type 2 diabetes mellitus, in particular a non-diabetic patient.

According to another embodiment this invention provides a method for treating chronic heart failure with reduced ejection fraction (HFrEF) in a patient in need thereof comprising administering empagliflozin to the patient, for example in a patient with chronic heart failure according to NYHA class I, II, III or IV. According to an aspect of this embodiment the extent of chronic heart failure in a patient with chronic heart failure according to NYHA class II, III or IV is improved according to the NYHA classification. The patient according to this embodiment is for example a non-diabetic patient, a patient with pre-diabetes or a patient with type 2 diabetes mellitus, in particular a non-diabetic patient.

According to an embodiment this invention provides a method for reducing the risk of cardiovascular death in a patient with chronic heart failure, for example according to NYHA class II, III or IV, with preserved ejection fraction (HFpEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to another embodiment this invention provides a method for reducing the risk of cardiovascular death in a patient with chronic heart failure, for example according to NYHA class II, III or IV, with reduced ejection fraction (HFrEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to an embodiment this invention provides a method for reducing the risk of hospitalization for heart failure in a patient with chronic heart failure, for example according to NYHA class II, III or IV, with preserved ejection fraction (HFpEF) comprising administering empagliflozin to the patient. According to an aspect of this embodiment the patient has chronic heart failure according to NYHA class I. According to an aspect of this embodiment the risk of first hospitalization for heart failure is reduced. According to another aspect of this embodiment the risk of re-hospitalization for heart failure is reduced. The patient according to this embodiment is for example a non-diabetic patient, a patient with pre-diabetes or a patient with type 2 diabetes mellitus, in particular a non-diabetic patient.

According to another embodiment this invention provides a method for reducing the risk of hospitalization for heart failure in a patient with chronic heart failure, for example according to NYHA class II, III or IV, with reduced ejection fraction (HFrEF) comprising administering empagliflozin to the patient. According to an aspect of this embodiment the patient has chronic heart failure according to NYHA class I. According to an aspect of this embodiment the risk of first hospitalization for heart failure is reduced. According to another aspect of this embodiment the risk of re-hospitalization for heart failure is reduced. The patient according to this embodiment is for example a non-diabetic patient, a patient with pre-diabetes or a patient with type 2 diabetes mellitus, in particular a non-diabetic patient.

According to an embodiment this invention provides a method for reducing the risk of cardiovascular death and hospitalization for heart failure in a patient with chronic heart failure, for example according to NYHA class II, III or IV, with preserved ejection fraction (HFpEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to another embodiment this invention provides a method for reducing the risk of cardiovascular death and hospitalization for heart failure in a patient with chronic heart failure, for example according to NYHA class II, III or IV, with reduced ejection fraction (HFrEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to an embodiment this invention provides a method for reducing all-cause mortality in a patient with chronic heart failure, for example according to NYHA class II, III or IV, with preserved ejection fraction (HFpEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to an embodiment this invention provides a method for reducing all-cause mortality in a patient with chronic heart failure, for example according to NYHA class II, III or IV, with reduced ejection fraction (HFrEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to an embodiment this invention provides a method for reducing the risk of all-cause hospitalization in a patient with chronic heart failure, for example according to NYHA class II, III or IV, with preserved ejection fraction (HFpEF) comprising administering empagliflozin to the patient. For example the patient has chronic heart failure according to NYHA class I. According to another aspect of this embodiment the risk of first all-cause hospitalization is reduced. According to another aspect of this embodiment the risk of recurrent all-cause hospitalization is reduced. The patient according to this embodiment is for example a non-diabetic patient, a patient with pre-diabetes or a patient with type 2 diabetes mellitus, in particular a non-diabetic patient.

According to another embodiment this invention provides a method for reducing the risk of all-cause hospitalization in a patient with chronic heart failure, for example according to NYHA class II, III or IV, with reduced ejection fraction (HFrEF) comprising administering empagliflozin to the patient. For example the patient has chronic heart failure according to NYHA class I. According to an aspect of this embodiment the risk of first all-cause hospitalization is reduced. According to another aspect of this embodiment the risk of recurrent all-cause hospitalization is reduced. The patient according to this embodiment is for example a non-diabetic patient, a patient with pre-diabetes or a patient with type 2 diabetes mellitus, in particular a non-diabetic patient.

According to an embodiment this invention provides a method for reducing the risk of new onset of atrial fibrillation in a patient with chronic heart failure, for example according to NYHA class II, III or IV, with preserved ejection fraction (HFpEF) comprising administering empagliflozin to the patient. For example the patient has chronic heart failure according to NYHA class I. The patient according to this embodiment is for example a non-diabetic patient, a patient with pre-diabetes or a patient with type 2 diabetes mellitus, in particular a non-diabetic patient.

According to another embodiment this invention provides a method for reducing the risk of new onset of atrial fibrillation in a patient with chronic heart failure, for example according to NYHA class II, III or IV, with reduced ejection fraction (HFrEF) comprising administering empagliflozin to the patient. For example the patient has chronic heart failure according to NYHA class I. The patient according to this embodiment is for example a non-diabetic patient, a patient with pre-diabetes or a patient with type 2 diabetes mellitus, in particular a non-diabetic patient.

According to an embodiment this invention provides a method for improving the health related quality of life and/or the functional capacity, in particular the exercise capacity, in a patient with chronic heart failure, for example according to NYHA class II, III or IV, with preserved ejection fraction (HFpEF) comprising administering empagliflozin to the patient. For example the patient has chronic heart failure according to NYHA class I. According to an aspect of this embodiment the health related quality of life is measured by a questionnaire such as for example KCCQ or KCCQ-12. According to another aspect of this embodiment the health related quality of life or exercise capacity is measured by a walk test, for example a 6 minutes walk test, or by the maximum oxygen uptake (VO₂max). The patient according to this embodiment is for example a non-diabetic patient, a patient with pre-diabetes or a patient with type 2 diabetes mellitus, in particular a non-diabetic patient.

According to an embodiment this invention provides a method for improving the health related quality of life and/or the functional capacity, in particular the exercise capacity in a patient with chronic heart failure, for example according to NYHA class II, III or IV, with reduced ejection fraction (HFrEF) comprising administering empagliflozin to the patient. For example the patient has chronic heart failure according to NYHA class I. According to an aspect of this embodiment the health related quality of life measured by a questionnaire such as for example KCCQ or KCCQ-12. According to another aspect of this embodiment the health related quality of life or exercise capacity is measured by a walk test, for example a 6 minutes walk test, or by the maximum oxygen uptake (VO₂max). The patient according to this embodiment is for example a non-diabetic patient, a patient with pre-diabetes or a patient with type 2 diabetes mellitus, in particular a non-diabetic patient.

According to an embodiment this invention provides a method for treating, preventing, protecting against, reducing the risk of or delaying the occurrence of acute decompensated heart failure (ADHF) in a patient with chronic heart failure, for example according to NYHA class II, III or IV, with preserved ejection fraction (HFpEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to another embodiment this invention provides a method for treating, preventing, protecting against, reducing the risk of or delaying the occurrence of acute decompensated heart failure (ADHF) reducing the risk of new onset of atrial fibrillation in a patient with chronic heart failure, for example according to NYHA class II, III or IV, with reduced ejection fraction (HFrEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to an embodiment this invention provides a method for reducing the risk of new onset of type 2 diabetes mellitus in a patient with chronic heart failure, for example according to NYHA class II, III or IV, with preserved ejection fraction (HFpEF) comprising administering empagliflozin to the patient. For example the patient has chronic heart failure according to NYHA class I. According to an aspect of this invention the patient is a non-diabetic patient. According to another aspect of this invention the patient is a patient with pre-diabetes.

According to an embodiment this invention provides a method for reducing the risk of new onset of type 2 diabetes mellitus in a patient with chronic heart failure, for example according to NYHA class II, III or IV, with reduced ejection fraction (HFrEF) comprising administering empagliflozin to the patient. For example the patient has chronic heart failure according to NYHA class I. According to an aspect of this invention the patient is a non-diabetic patient. According to another aspect of this invention the patient is a patient with pre-diabetes.

According to an embodiment this invention provides a method for reducing the risk of myocardial infarction in a patient with chronic heart failure, for example according to NYHA class II, III or IV, with preserved ejection fraction (HFpEF) comprising administering empagliflozin to the patient. For example the patient has chronic heart failure according to NYHA class I. According to an aspect of this embodiment the risk on non-fatal myocardial infarction is reduced. According to an aspect of this embodiment the risk on fatal myocardial infarction is reduced. The patient according to this embodiment is for example a non-diabetic patient, a patient with pre-diabetes or a patient with type 2 diabetes mellitus, in particular a non-diabetic patient.

According to an embodiment this invention provides a method for reducing the risk of myocardial infarction in a patient with chronic heart failure, for example according to NYHA class II, III or IV, with reduced ejection fraction (HFrEF) comprising administering empagliflozin to the patient. For example the patient has chronic heart failure according to NYHA class I. According to an aspect of this embodiment the risk on non-fatal myocardial infarction is reduced. According to an aspect of this embodiment the risk on fatal myocardial infarction is reduced. The patient according to this embodiment is for example a non-diabetic patient, a patient with pre-diabetes or a patient with type 2 diabetes mellitus, in particular a non-diabetic patient.

According to an embodiment this invention provides a method for reducing the risk of stroke in a patient with chronic heart failure, for example according to NYHA class II, III or IV, with preserved ejection fraction (HFpEF) comprising administering empagliflozin to the patient. For example the patient has chronic heart failure according to NYHA class I. According to an aspect of this embodiment the risk on non-fatal stroke is reduced. According to an aspect of this embodiment the risk on fatal stroke is reduced. The patient according to this embodiment is for example a non-diabetic patient, a patient with pre-diabetes or a patient with type 2 diabetes mellitus, in particular a non-diabetic patient.

According to an embodiment this invention provides a method for reducing the risk of stroke in a patient with chronic heart failure, for example according to NYHA class II, III or IV, with reduced ejection fraction (HFrEF) comprising administering empagliflozin to the patient. For example the patient has chronic heart failure according to NYHA class I. According to an aspect of this embodiment the risk on non-fatal stroke is reduced. According to an aspect of this embodiment the risk on fatal stroke is reduced. The patient according to this embodiment is for example a non-diabetic patient, a patient with pre-diabetes or a patient with type 2 diabetes mellitus, in particular a non-diabetic patient.

According to an embodiment this invention provides a method for reducing the risk of any of cardiovascular death, non-fatal myocardial infarction, non-fatal stroke (the so-called 3-point MACE) in a patient with chronic heart failure, for example according to NYHA class II, III or IV, with preserved ejection fraction (HFpEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to an embodiment this invention provides a method for reducing the risk of any of cardiovascular death (including fatal stroke, fatal myocardial infarction and sudden death), non-fatal myocardial infarction (excluding silent myocardial infarction), non-fatal stroke (the so-called 3-point MACE) in a patient with chronic heart failure, for example according to NYHA class II, III or IV, with preserved ejection fraction (HFpEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to an embodiment this invention provides a method for reducing the risk of any of cardiovascular death, non-fatal myocardial infarction, non-fatal stroke (the so-called 3-point MACE) in a patient with chronic heart failure, for example according to NYHA class II, III or IV, with reduced ejection fraction (HFrEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to an embodiment this invention provides a method for reducing the risk of any of cardiovascular death (including fatal stroke, fatal myocardial infarction and sudden death), non-fatal myocardial infarction (excluding silent myocardial infarction), non-fatal stroke (the so-called 3-point MACE) in a patient with chronic heart failure, for example according to NYHA class II, III or IV, with reduced ejection fraction (HFrEF) comprising administering empagliflozin to the patient. According to an aspect of this embodiment the patient has chronic heart failure according to NYHA class I. The patient according to this embodiment is for example a non-diabetic patient, a patient with pre-diabetes or a patient with type 2 diabetes mellitus, in particular a non-diabetic patient.

According to an embodiment this invention provides a method for preventing, slowing or reversing the progression to macroalbuminuria in a patient with chronic heart failure, for example according to NYHA class II, III or IV, with preserved ejection fraction (HFpEF) comprising administering empagliflozin to the patient. For example the patient has chronic heart failure according to NYHA class I. According to an aspect of this embodiment the progression from microalbuminuria to macroalbuminuria is prevented, slowed or reversed. The patient according to this embodiment is for example a non-diabetic patient, a patient with pre-diabetes or a patient with type 2 diabetes mellitus, in particular a non-diabetic patient.

According to an embodiment this invention provides a method for preventing, slowing or reversing the progression to macroalbuminuria in a patient with chronic heart failure, for example according to NYHA class II, III or IV, with reduced ejection fraction (HFrEF) comprising administering empagliflozin to the patient. For example the patient has chronic heart failure according to NYHA class I. According to an aspect of this embodiment the progression from microalbuminuria to macroalbuminuria is prevented, slowed or reversed. The patient according to this embodiment is for example a non-diabetic patient, a patient with pre-diabetes or a patient with type 2 diabetes mellitus, in particular a non-diabetic patient.

According to an embodiment this invention provides a method for improving the renal function or for renal protection in a patient with chronic heart failure, for example according to NYHA class II, III or IV, with preserved ejection fraction (HFpEF) comprising administering empagliflozin to the patient. For example the patient has chronic heart failure according to NYHA class I. According to an aspect of this embodiment the patient has mild,moderate or severe renal impairment. The patient according to this embodiment is for example a non-diabetic patient, a patient with pre-diabetes or a patient with type 2 diabetes mellitus, in particular a non-diabetic patient. According to an aspect of this embodiment the improvement of the renal function or the renal protection is a slowing of a decline in eGFR, for example a slowing of a progressive decline in eGFR or a slowing of a natural progressive decline in eGFR. According to another aspect of this embodiment the improvement of the renal function or the renal protection is diagnosed by an improvement of the eGFR.

According to an embodiment this invention provides a method for improving the renal function or for renal protection in a patient with chronic heart failure, for example according to NYHA class II, III or IV, with reduced ejection fraction (HFrEF) comprising administering empagliflozin to the patient. For example the patient has chronic heart failure according to NYHA class I. According to an aspect of this embodiment the patient has mild, moderate or severe renal impairment. The patient according to this embodiment is for example a non-diabetic patient, a patient with pre-diabetes or a patient with type 2 diabetes mellitus, in particular a non-diabetic patient. According to an aspect of this embodiment the improvement of the renal function or the renal protection is a slowing of a decline in eGFR, for example a slowing of a progressive decline in eGFR or a slowing of a natural progressive decline in eGFR. According to an aspect of this embodiment the improvement of the renal function or the renal protection is diagnosed by an improvement of the eGFR.

According to an embodiment this invention provides a method for treating, preventing, protecting against, reducing the risk of, delaying the occurrence of and/or delaying the progression of chronic kidney disease in a patient diagnosed with chronic heart failure comprising administering empagliflozin to the patient. In particular this embodiment relates to a method for treating and/or delaying the progression of chronic kidney disease in a patient diagnosed with chronic heart failure comprising administering empagliflozin to the patient. According to another aspect of this embodiment the patient is a patient with stage 2 chronic kidney disease. According to an aspect of this embodiment the patient is a patient with stage 3, including stage 3a and/or 3b, chronic kidney disease. According to another aspect of this embodiment the patient is a patient with stage 4 chronic kidney disease. According to an aspect of this embodimemt the patient is a patient with stage 3, including stage 3a and/or 3b, or stage 4 chronic kidney disease and with chronic heart failure, for example according to NYHA class I, II, III or IV, with preserved ejection fraction (HFpEF). According to another aspect of this embodiment the patient is a patient with stage 2 chronic kidney disease. According to another aspect of this embodiment the patient is a patient with stage 3, including stage 3a and/or 3b, or stage 4 chronic kidney disease and with chronic heart failure, for example according to NYHA class I, II, III or IV, with reduced ejection fraction (HFrEF). The patient according to this embodiment, including the various aspects of this embodiment, is for example a non-diabetic patient, a patient with pre-diabetes or a patient with type 2 diabetes mellitus, in particular a non-diabetic patient.

According to another embodiment this invention provides a method for treating, preventing, protecting against, reducing the risk of, delaying the occurrence of and/or delaying the progression of chronic kidney disease in a patient not diagnosed with chronic heart failure comprising administering empagliflozin to the patient wherein the patient is a non-diabetic patient. In particular this embodiment relates to a method for treating and/or delaying the progression of chronic kidney disease in the patient. According to an aspect of this embodiment the patient is a patient with stage 3, including stage 3a and/or 3b, chronic kidney disease. According to another aspect of this embodiment the patient is a patient with stage 4 chronic kidney disease.

In one embodiment, the present invention provides a method of treating, preventing, protecting against or delaying the occurrence of:
new onset of albuminuria,
progression from no albuminuria to micro- or macroalbuminuria,
doubling of serum creatinine level accompanied by an eGFR (based on modification of diet in renal disease (MDRD) formula) ≤45 mL/min/1.73 m²,
sustained reduction of ≥30%, ≥40%, ≥50% or ≥57% eGFR (CKD-EPI), in particular sustained reduction of ≥40% eGFR (CKD-EPI),
sustained eGFR (CKD-EPI)<15 mL/min/1.73 m² for patients with baseline eGFR≥30 mL/min/1.73 m²,
sustained eGFR (CKD-EPI)<10 mL/min/1.73 m² for patients with baseline eGFR<30 mL/min/1.73 m²,
need for continuous renal replacement therapy,
need for chronic dialysis treatment,
need for receiving a renal transplant,
death due to renal disease, or
composite of sustained reduction of ≥40% eGFR (CKD-EPI) or sustained eGFR (CKD-EPI)<15 mL/min/1.73 m² for patients with baseline eGFR≥30 mL/min/1.73 m², and
sustained eGFR (CKD-EPI)<10 mL/min/1.73 m² for patients with baseline eGFR<30 mL/min/1.73 m², or
composite of sustained reduction of ≥40% eGFR (CKD-EPI) or sustained eGFR (CKD-EPI)<15 mL/min/1.73 m² for patients with baseline eGFR≥30 mL/min/1.73 m²,
sustained eGFR (CKD-EPI)<10 mL/min/1.73 m² for patients with baseline eGFR<30 mL/min/1.73 m²,
need for chronic dialysis treatment, and
need for receiving a renal transplant.

in a patient diagnosed with chronic heart failure, said method comprising administering empagliflozin to the patient. According to an aspect of this embodimemt the patient is a patient with chronic heart failure, for example according to NYHA class I, II, III or IV, with preserved ejection fraction (HFpEF). According to another aspect of this embodiment the patient is a patient with chronic heart failure, for example according to NYHA class I, II, III or IV, with reduced ejection fraction (HFrEF). The patient according to this embodiment, including the various aspects of this embodiment, is for example a non-diabetic patient, a patient with pre-diabetes or a patient with type 2 diabetes mellitus, in particular a non-diabetic patient.

In the methods according to the present invention empagliflozin is optionally administered in combination with one or more other therapeutic substances to the patient.

According to one embodiment of the methods as described hereinbefore and hereinafter the patient is a patient with an elevated BNP or an elevated plasma NT-proBNP. For example the patient has an elevated BNP of equal to or greater than 75 pg/mL (NT-proBNP 300 pg/mL) or equal to or greater than 100 pg/mL (NT-proBNP≥400 pg/mL) or equal to or greater than 150 pg/mL (NT-proBNP≥600 pg/mL) or equal to or greater than 225 pg/mL (NT-proBNP 900 pg/mL).

According to another embodiment of the methods as described hereinbefore and hereinafter the patient is a patient who was hospitalized for heart failure within the last 9 months, in particular hospitalized for heart failure within the last 9 months and has an elevated BNP or NT-proBNP.

According to an embodiment of the methods as described hereinbefore and hereinafter the patient is a patient with reduced ejection fraction (HFrEF) and an ejection fraction EF≥36% to ≤40% and an elevated NT-proBNP≥2500 pg/ml for patients without atrial fibrillation, or ≥5000 pg/ml for patients with atrial fibrillation.

According to an embodiment of the methods as described hereinbefore and hereinafter the patient is a patient with reduced ejection fraction (HFrEF) and an ejection fraction EF≥31% to ≤35% and an elevated NT-proBNP≥1000 pg/ml for patients without atrial fibrillation, or ≥2000 pg/ml for patients with atrial fibrillation.

According to an embodiment of the methods as described hereinbefore and hereinafter the patient is a patient with reduced ejection fraction (HFrEF) and an ejection fraction EF≤30% and an elevated NT-proBNP≥600 pg/ml for patients without atrial fibrillation, or ≥200 pg/ml for patients with atrial fibrillation.

According to one embodiment of the methods as described hereinbefore and hereinafter the patient is a patient with normal renal function or with mild renal impairment or with moderate renal or severe renal impairment. According to this embodiment the patient has an eGFR equal to or greater than 20 mL/min/1.73 m² .

According to one embodiment of the methods as described hereinbefore and hereinafter the patient is a patient with normal renal function or with mild renal impairment or with moderate renal impairment. According to this embodiment the patient has an eGFR equal to or greater than 30 mL/min/1.73 m² .

According to another embodiment of the methods as described hereinbefore and hereinafter the patient is a patient with normal renal function or with mild renal impairment or with moderate A renal impairment (CKD 3A). According to this embodiment the patient has an eGFR equal to or greater than 45 mL/min/1.73 m² .

According to another embodiment of the methods as described hereinbefore and hereinafter the patient is a patient with normal renal function or with mild renal impairment. According to this embodiment the patient has an eGFR equal to or greater than 60 mL/min/1.73 m² .

According to another embodiment of the methods as described hereinbefore and hereinafter the patient is a patient with moderate A renal impairment (CKD 3A). According to this embodiment the patient has an eGFR equal to or greater than 45 and lower than 60 mL/min/1.73 m² .

According to another embodiment of the methods as described hereinbefore and hereinafter the patient is a patient with moderate B renal impairment (CKD 3B). According to this embodiment the patient has an eGFR equal to or greater than 30 and lower than 45 mL/min/1.73 m² .

According to an embodiment of the methods as described hereinbefore and hereinafter the patient is a non-diabetic patient, a patient with pre-diabetes, a patient with type 2 diabetes mellitus or a patient with type 1 diabetes mellitus.

According to another embodiment of the methods as described hereinbefore and hereinafter the patient is a non-diabetic patient, a patient with pre-diabetes or a patient with type 2 diabetes mellitus.

According to another embodiment of the methods as described hereinbefore and hereinafter the patient is a patient with pre-diabetes. According to an aspect of this embodiment the patient has a HbA1c more or equal to 5.7% and less than 6.5%.

According to another embodiment of the methods as described hereinbefore and hereinafter the patient is a patient with pre-diabetes or a non-diabetic patient. According to an aspect of this embodiment the patient has a HbA1c less than 6.5%.

According to another embodiment of the methods as described hereinbefore and hereinafter the patient is a non-diabetic patient. According to an aspect of this embodiment the patient has a HbA1c less than 5.7%.

According to another aspect the non-diabetic patient does not show an impaired glucose tolerance (IGT), i.e. the patient shows a normal glucose tolerance. For example the 2 hour postprandial blood glucose or plasma glucose (PG) concentration is smaller than 140 mg/dl (7.78 mmol/L).

According to another aspect the non-diabetic patient does not show an impaired fasting blood glucose (IFG), i.e. the patient shows a normal fasting glucose. For example the fasting plasma glucose concentration (FPG) is smaller than 100 mg/dl, i.e. smaller than 5.6 mmol/l.

In particular the non-diabetic patient does not show an impaired fasting blood glucose (IFG) and does not show an impaired glucose tolerance (IGT), i.e. the patient shows a normal glucose tolerance and a normal glucose tolerance. For example the fasting plasma glucose concentration (FPG) is smaller than 100 mg/dl, i.e. smaller than 5.6 mmol/l, and the 2 hour postprandial blood glucose or plasma glucose (PG) concentration is smaller than 140 mg/dl (7.78 mmol/L).

According to an embodiment of the methods as described hereinbefore and hereinafter empagliflozin is administered at a dose in a range from 1 to 25 mg per day, for example at a dose of 1 mg, 2.5 mg, 5 mg, 7.5 mg, 10 mg or 25 mg per day to the patient. The administration of empagliflozin may occur one or two times a day, most preferably once a day. For example a dose for once daily administration is 10 mg or 25 mg. The preferred route of administration is oral administration.

According to a particular aspect of the present invention empagliflozin is administered at a dose of 10 mg per day to the patient.

According to another particular aspect of the present invention empagliflozin is administered at a dose of 25 mg per day to the patient.

Preferably empagliflozin is administered orally to the patient once daily.

In one embodiment, patients within the meaning of this invention may include patients with chronic heart failure who have not previously been treated with a drug to treat chronic heart failure (heart-failure-drug-naïve patients). Thus, in an embodiment, the therapies described herein may be used in heart-failure-drug-naïve patients.

In another embodiment, patients within the meaning of this invention may include patients with chronic heart failure and with pre-diabetes or with type 2 diabetes mellitus (T2DM) who have not previously been treated with an antidiabetic drug (T2DM-drug-naïve patients). Thus, in an embodiment, the therapies described herein may be used in T2DM-drug-naïve patients.

Furthermore, the methods according to this invention are particularly suitable in the treatment of patients with chronic heart failure and with insulin dependency, i.e. in patients who are treated or otherwise would be treated or need treatment with an insulin or a derivative of insulin or a substitute of insulin or a formulation comprising an insulin or a derivative or substitute thereof. These patients include patients with diabetes type 2 and patients with diabetes type 1.

Furthermore, it can be found that the administration of a pharmaceutical composition according to this invention results in no risk or in a low risk of hypoglycemia. Therefore, a treatment or prophylaxis according to this invention is also advantageously possible in those patients showing or having an increased risk for hypoglycemia.

By the administration of empagliflozin excessive blood glucose is excreted through the urine of the patient based on the SGLT2 inhibiting activity, so that no gain in weight or even a reduction in body weight of the patient may result. Therefore, the methods according to this invention are advantageously suitable in those patients with chronic heart failure who are diagnosed of one or more of the conditions selected from the group consisting of overweight and obesity, in particular class I obesity, class II obesity, class III obesity, visceral obesity and abdominal obesity. In addition a method according to this invention is advantageously suitable in those patients in which a weight increase is contraindicated.

When this invention refers to patients requiring treatment or prevention, it relates primarily to treatment and prevention in humans, but the pharmaceutical composition may also be used accordingly in veterinary medicine in mammals. In the scope of this invention adult patients are preferably humans of the age of 18 years or older. Also in the scope of this invention, patients are adolescent humans, i.e. humans of age 10 to 17 years, preferably of age 13 to 17 years.

According to an embodiment of the present invention empagliflozin is administered in combination with one or more other therapeutic substances to the patient. The combined administration may be simultaneously, separately or sequentially.

In one aspect of this embodiment of the present invention, the one or more other therapeutic substances are selected from active substances that are indicated in the treatment of chronic heart failure, antidiabetic substances, active substances that lower the total cholesterol, LDL-cholesterol, Non-HDL-cholesterol and/or Lp(a) level in the blood, active substances that raise the HDL-cholesterol level in the blood, active substances that lower blood pressure, active substances that are indicated in the treatment of atherosclerosis or obesity, antiplatelet agents, anticoagulant agents, and vascular endothelial protective agents.

In one embodiment, the active substances that are indicated in the treatment of chronic heart failure are selected from angiotensin receptor blockers (ARB), angiotensin-converting enzyme (ACE) inhibitors, angiotensin receptor neprilysin inhibitors (ARNi), beta-blockers, aldosterone antagonists (MRA), digoxin, ivabradine and diuretics.

In one embodiment, the antidiabetic substances are selected from metformin, sulphonylureas, nateglinide, repaglinide, PPAR-gamma agonists, alpha-glucosidase inhibitors, insulin and insulin analogues, GLP-1 and GLP-1 analogues and DPP-4 inhibitors.

In one embodiment the patient receives standard of care, which includes medication and/or devices indicated for patients with heart failure, such as chronic or acute heart failure. In one aspect the patient, in particular diagnosed with HFrEF, has or receives a device selected from the group of ICD (implantable cardioverter defibrillator) and CRT (cardiac resynchronization therapy), such as CRT-P (CRT pacemaker) and CRT-D (CRT combination of pacemaker and defibrillator).

In one embodiment the patient receives standard of care medication indicated for patients with chronic heart failure. In one aspect of this embodiment empagliflozin is administered to the patient in combination with one or more active substances that are indicated in the treatment of chronic heart failure. For example empagliflozin is adminstered in combination with one or more active substances selected from the group consisting of angiotensin receptor blockers (ARB), angiotensin-converting enzyme (ACE) inhibitors, beta-blockers, aldosterone antagonists, diuretics, angiotensin receptor-neprilysin inhibitor (ARNi), mineralcorticoid receptor antagonists and ivabradine. According to this aspect of the embodiment the patient is for example a non-diabetic patient or a patient with pre-diabetes.

In one aspect of this embodiment, the number, dosage and/or regimen of said medications to treat chronic heart failure is reduced in said patient, while the administration of empagliflozin is continued. For example the dose of one or more diuretics administered to the patient may be reduced, while the administration of empagliflozin is continued.

Examples of angiotensin II receptor blockers (ARBs) are telmisartan, candesartan, valsartan, losartan, irbesartan, olmesartan, azilsartan and eprosartan; the dosage(s) of some of these medications are for example shown below:
Candesartan (Atacand), 4 mg, 8 mg, 16 mg, or 32 mg of candesartan cilexetil
Eprosartan (Teveten), 400 mg or 600 mg
Irbesartan (Avapro), 75 mg, 150 mg, or 300 mg of irbesartan.
Losartan (Cozaar), 25 mg, 50 mg or 100 mg of losartan potassium
Telmisartan (Micardis), 40 mg or 80 mg
Telmisartan (Micardis HCT), 40 mg/12.5 mg, 80 mg/12.5 mg, and 80 mg/25 mg each of telmisartan and hydrochlorothiazide
Telmisartan/amlodipine (Twynsta), 40 mg/5 mg, 40 mg/10 mg, 80 mg/5 mg and 80 mg/10 mg each of telmisartan and amlodipine
Valsartan (Diovan), 40 mg, 80 mg, 160 mg or 320 mg of valsartan

Examples of Angiotensin-Converting Enzyme (ACE) inhibitors are benazepril, captopril, ramipril, lisinopril, Moexipril, cilazapril, quinapril, captopril, enalapril, benazepril, perindopril, fosinopril and trandolapril; the dosage(s) of some of these medications are for example shown below:
Benazepril (Lotensin), 5 mg, 10 mg, 20 mg, and 40 mg for oral administration
Captopril (Capoten), 12.5 mg, 25 mg, 50 mg, and 100 mg as scored tablets for oral administration
Enalapril (Vasotec), 2.5 mg, 5 mg, 10 mg, and 20 mg tablets for oral administration
Fosinopril (Monopril), for oral administration as 10 mg, 20 mg, and 40 mg tablets
Lisinopril (Prinivil, Zestril), 5 mg, 10 mg, and 20 mg tablets for oral administration
Moexipril (Univasc), 7.5 mg and 15 mg for oral administration
Perindopril (Aceon), 2 mg, 4 mg and 8 mg strengths for oral administration
Quinapril (Accupril), 5 mg, 10 mg, 20 mg, or 40 mg of quinapril for oral administration
Ramipril (Altace), 1.25 mg, 2.5 mg, 5, mg, 10 mg
Trandolapril (Mavik) , 1 mg, 2 mg, or 4 mg of trandolapril for oral administration

Examples of beta-blockers are acebutolol, atenolol, betaxolol, bisoprolol, celiprolol, metoprolol, nebivolol, propranolol, timolol and carvedilol; the dosage(s) of some of these medications are for example shown below:
Acebutolol (Sectral), 200 or 400 mg of acebutolol as the hydrochloride salt
Atenolol (Tenormin), 25, 50 and 100 mg tablets for oral administration
Betaxolol (Kerlone), 10-mg and 20-mg tablets for oral administration
Bisoprolol/hydrochlorothiazide (Ziac), 2.5/6 mg, 5/6.25 mg, 10/6.25 mg
Bisoprolol (Zebeta), 5 and 10 mg tablets for oral administration
Metoprolol (Lopressor, Toprol XL), 50- and 100-mg tablets for oral administration and in 5-mL ampuls for intravenous administration
Propranolol (Inderal), 10 mg, 20 mg, 40 mg, 60 mg, and 80 mg tablets for oral administration
Timolol (Blocadren), 5 mg, 10 mg or 20 mg timolol maleate for oral administration.

Examples of aldosterone antagonists are spironolactone, eplerenone, canrenone and fineronone; the dosage(s) of some of these medications are for example shown below:
spironolactone (e.g. Aldactone), 25 or 50 mg once daily or every second day,
eplerenone (e.g. Inspra), 25 or 50 mg once daily.

Examples of diuretics are bumetanide, hydrochlorothiazide, chlortalidon, chlorothiazide, hydrochlorothiazide, xipamide, indapamide, furosemide, piretanide, torasemide, spironolactone, eplerenone, amiloride and triamterene; for example these medications are thiazide diuretics, e.g. chlorthalidone, HCT, loop diuretics, e.g. furosemide, torasemide or potassium-sparing diuretics, e.g. eplerenone, or combination thereof; the dosage(s) of some of these medications are for example shown below:
Amiloride (Midamor), 5 mg of anhydrous amiloride HCl
Bumetanide (Bumex), available as scored tablets, 0.5 mg (light green), 1 mg (yellow) and 2 mg (peach) for oral administration
Chlorothiazide (Diuril),
Chlorthalidone (Hygroton)
Furosemide (Lasix)
Hydro-chlorothiazide (Esidrix, Hydrodiuril)
Indapamide (Lozol) and Spironolactone (Aldactone)
Eplerenone (Inspra)

An example of an angiotensin receptor-neprilysin inhibitor (ARNi) is a combination of valsartan and sacubitril (Entresto).

An example of inhibition of the cardiac pacemaker If current is ivabradine (Procoralan, Corlanor).

Examples of calcium channel blockers are amlodipine, nifedipine, nitrendipine, nisoldipine, nicardipine, felodipine, lacidipine, lercanipidine, manidipine, isradipine, nilvadipine, verapamil, gallopamil and diltiazem.

Examples of medications that lower blood pressure include angiotensin II receptor blockers (ARBs), Angiotensin-Converting Enzyme (ACE) inhibitors, beta-blockers, diuretics and calcium channel blockers.

In another aspect of this embodiment the patient is a patient with type 2 diabetes mellitus and empagliflozin is administered to the patient in combination with one or more active substances that are indicated in the treatment of chronic heart failure and in combination with one or more antidiabetic substances. The antidiabetic substances include metformin, sulphonylureas, nateglinide, repaglinide, PPAR-gamma agonists, alpha-glucosidase inhibitors, insulin and insulin analogues, GLP-1 and GLP-1 analogues and DPP-4 inhibitors. Examples thereof are metformin and DPPIV inhibitors, such as sitagliptin, saxaglitpin and linagliptin. The active substances that are indicated in the treatment of chronic heart failure include angiotensin receptor blockers (ARB), angiotensin-converting enzyme (ACE) inhibitors, beta-blockers, aldosterone antagonists and diuretics.

Therefore according to one aspect of the methods according to this invention empagliflozin is administered in combination with linagliptin to the patient. The patient according to this aspect is in particular a patient with type 2 diabetes mellitus. Preferred doses are for example 10 mg empagliflozin once daily and 5 mg linagliptin once daily.

Therefore according to another aspect of the methods according to this invention empagliflozin is administered in combination with metformin hydrochloride to the patient. The patient according to this aspect is in particular a patient with type 2 diabetes mellitus.

Preferred doses are for example 10 mg empagliflozin once daily or 5 mg empagliflozin twice daily and 500 mg, 850 mg or 1000 mg metformin hydrochloride twice daily.

In one aspect of this embodiment, the number, dosage and/or regimen of said medications to treat chronic heart failure is reduced in said patient, while the administration of empagliflozin is continued. In another aspect of this embodiment, the number, dosage and/or regimen of said medications to treat type 2 diabetes mellitus is reduced in said patient, while the administration of empagliflozin is continued. In yet another aspect of this embodiment, the numbers, dosages and/or regimens of said medications to treat type 2 diabetes mellitus and of said medications to treat chronic heart failure are reduced in said patient, while the administration of empagliflozin is continued.

According to an example of this aspect empagliflozin is adminstered in combination with one or more active substances selected from the group consisting of angiotensin receptor blockers (ARB), angiotensin-converting enzyme (ACE) inhibitors, beta-blockers, aldosterone antagonists, diuretics, angiotensin receptor-neprilysin inhibitor (ARNi), mineralcorticoid receptor antagonists and ivabradine in combination with metformin or in combination with linagliptin or in combination of metformin and linagliptin.

Examples of active substances in the above described groups are known to the one skilled in the art, including their dose strengths, administration schemes and formulations.

In the context of this invention the term metformin comprises metformin hydrochloride in the form of an immediate release, extended or slow release formulation. Doses of metformin hydrochloride administered to the patient are particularly 500 mg to 2000 mg per day, for example 750 mg, 1000 mg, 1500 and 2000 mg per day.

Empagliflozin and metformin may be adminstered separately in two different dosage forms or combined in one dosage form. Combined dosage forms of empagliflozin and metformin as immediate release formulations are described in WO 2011/039337 and are known for example as SYNJARDI®. Combined dosage forms of empagliflozin and metformin wherein empagliflozin is part of an immediate release formulation and metformin is part of an extended release formulation are described in WO 2012/120040 and WO 2013/131967.

A preferred dose of linagliptin administered to the patient is 5 mg per day.

Empagliflozin and linagliptin may be adminstered separately in two different dosage forms or combined in one dosage form. Combined dosage forms of empagliflozin and linagliptin are described in WO 2010/092124 and are known for example as GLYXAMBI®.

Within this invention it is to be understood that the combinations, compositions or administrations in combination according to this invention may envisage the simultaneous, sequential or separate administration of the active components or ingredients.

In this context, “combination” or “combined” within the meaning of this invention may include, without being limited, fixed and non-fixed (e.g. free) forms (including kits) and uses, such as e.g. the simultaneous, sequential or separate use of the components or ingredients.

The combined administration of this invention may take place by administering the active components or ingredients together, such as e.g. by administering them simultaneously in one single or in two separate formulations or dosage forms. Alternatively, the administration may take place by administering the active components or ingredients sequentially, such as e.g. successively in two separate formulations or dosage forms.

For the combination therapy of this invention the active components or ingredients may be administered separately (which implies that they are formulated separately) or formulated altogether (which implies that they are formulated in the same preparation or in the same dosage form). Hence, the administration of one element of the combination of the present invention may be prior to, concurrent to, or subsequent to the administration of the other element of the combination.

Unless otherwise noted, combination therapy may refer to first line, second line or third line therapy, or initial or add-on combination therapy or replacement therapy.

The methods according to this invention are particularly suitable in the long term treatment or prophylaxis of the diseases and/or conditions as described hereinbefore and hereinafter. The term “long term” as used hereinbefore and hereinafter indicates a treatment of or administration in a patient within a period of time longer than 12 weeks, preferably longer than 25 weeks, even more preferably longer than 1 year.

The pharmaceutical composition comprising empagliflozin according to the invention may be formulated for oral or parenteral (including intramuscular, sub-cutaneous and intravenous) administration in liquid or solid form or in a form suitable for administration by inhalation or insufflation. Oral administration is preferred. The pharmaceutical composition may be formulated in the form of tablets, granules, fine granules, powders, capsules, caplets, soft capsules, pills, oral solutions, syrups, dry syrups, chewable tablets, troches, effervescent tablets, drops, suspension, fast dissolving tablets, oral fast-dispersing tablets, etc. The pharmaceutical composition and the dosage forms preferably comprises one or more pharmaceutical acceptable carriers which must be “acceptable” in the sense of being compatible with the other ingredients of the formulation and not deleterious to the recipient thereof. Examples of pharmaceutically acceptable carriers are known to the one skilled in the art.

The pharmaceutical compositions and methods according to this invention show advantageous effects in the treatment and prevention of those diseases and conditions as described hereinbefore. Advantageous effects may be seen for example with respect to efficacy, dosage strength, dosage frequency, pharmacodynamic properties, pharmacokinetic properties, fewer adverse effects, convenience, compliance, etc.

Methods for the manufacture of empagliflozin are known to the one skilled in the art. Advantageously, the compounds according to this invention can be prepared using synthetic methods as described in the literature, including patent applications as cited hereinbefore. Preferred methods of manufacture are described in the WO 2006/120208 and WO 2007/031548. With regard to empagliflozin an advantageous crystalline form is described in the international patent application WO 2006/117359 which hereby is incorporated herein in its entirety.

Further embodiments, features and advantages of the present invention may become apparent from the following examples. The following examples serve to illustrate, by way of example, the principles of the invention without restricting it.

### EXAMPLES

### Example 1

### Treatment of Patients with Chronic Heart Failure and HFrEF

The longterm impact on cardiovascular death or hospitalization for heart failure and other parameters of treatment with empagliflozin in a relevant population of patients with chronic heart failure and reduced ejection fraction is investigated as follows:

Patients with chronic heart failure and symptoms according to NYHA II, III or IV and a reduced ejection fraction (LVEF smaller or equal than 40%) and an elevated BNP (or elevated NT-proBNP), e.g. as defined below, are treated over a long term (e.g. for between approximately 20 to 38 months for each patient) with empagliflozin (optionally in combination with one or more other active substances, e.g. such as those described herein) and compared with patients who have been treated with a placebo on standard of care background medication.

Empagliflozin is administered orally once daily (for example 10 mg/daily). Patients include non-diabetic patients, patients with pre-diabetes and patients with type 2 diabetes mellitus. Pre-diabetes is diagnosed if HbA1c is more or equal to 5.7% and less than 6.5%. An individual is a non-diabetic patient if the HbA1c is less than 5.7%. Patients have a LVEF smaller or equal than 40%.

Patients with an elevated BNP (or elevated NT-proBNP) are defined as having one of the following:
an elevated BNP≥150 pg/mL or NT-proBNP≥600 pg/mL; or
if patients were hospitalized for heart failure within the last 9 months, an elevated BNP≥100 pg/mL or NT-proBNP≥400 pg/mL.

Patients with reduced ejection fraction may be included according to at least one of the following evidence of heart failure:

If the ejection fraction EF is ≥36% to ≤40% then the elevated NT-proBNP shall be ≥2500 pg/ml for patients without atrial fibrillation, or ≥5000 pg/ml for patients with atrial fibrillation.

If the ejection fraction EF is ≥31% to ≤35% then the elevated NT-proBNP shall be ≥1000 pg/ml for patients without atrial fibrillation, or ≥2000 pg/ml for patients with atrial fibrillation.

If the ejection fraction EF is ≤30% then the elevated NT-proBNP shall be ≥600 pg/ml for patients without atrial fibrillation, or ≥200 pg/ml for patients with atrial fibrillation.

The study is event-driven and all randomised patients will remain in the trial until the defined number of patients with primary endpoint events has been reached. The number of confirmed adjudicated primary endpoint events will be continuously monitored during the study.

The patients with cardiovascular risk factors are treated according to standard of care, which includes for example treatment with therapeutic agents selected from diuretics, ARNi, ACEi, ARB, statins, aspirin, beta-blockers, mineral corticoid antagonist or ivabradine,with or without cardiac device therapy including ICD, CRT-D or CRT-P.

Patients in the study follow the following criteria:
age above 18 years
Diagnosis of heart failure (HF). Definition of HF for inclusion in the study is left ventricular ejection fraction (LVEF) (ideally obtained by echocardiography, although radionuclide ventriculography and angiography are acceptable) smaller or equal than 40% (per local reading). The ejection fraction value is preferably obtained within 6 months prior to randomization and after any Myocardial Infarction (MI) or other event that would affect the ejection fraction.
Symptom(s) of heart failure (HF) (NYHA class II-IV)
At least one of the following: an elevated NT-proBNP≥600 pg/mL and/or if the patient was hospitalized for heart failure within the last 9 months an elevated NT-proBNP≥400 pg/mL.
Background therapy for heart failure if needed
Antidiabetic background if needed
Body Mass Index (BMI)<45 kg/m²
eGFR≥20 mL/min/1.73 m² or eGFR≥30 mL/min/1.73 m²

The time to cardiovascular death or hospitalisation for heart failure is determined in patients with heart failure with reduced ejection fraction (according to the criteria as described hereinbefore) treated with empagliflozin (e.g. 10 mg once daily) to placebo.

One or more of the following events are determined:
time to first hospitalisation for heart failure
eGFR (CKD-EPI) slope of change from baseline
Time to first occurrence of sustained reduction of ≥40% eGFR (CKD-EPI)
Time to first occurrence of sustained eGFR (CKD-EPI)<15 mL/min/1.73 m² for patients with baseline eGFR≥30 mL/min/1.73 m²
Time to first occurrence of sustained eGFR (CKD-EPI)<10 mL/min/1.73 m² for patients with baseline eGFR<30 mL/min/1.73 m²
Composite of time to first occurence of sustained reduction of ≥40% eGFR (CKD-EPI) or sustained eGFR (CKD-EPI)<15 mL/min/1.73 m² for patients with baseline eGFR≥30 mL/min/1.73 m², sustained eGFR (CKD-EPI)<10 mL/min/1.73 m² for patients with baseline eGFR<30 mL/min/1.73 m²,
Composite of time to first occurence of sustained reduction of ≥40% eGFR (CKD-EPI) or sustained eGFR (CKD-EPI)<15 mL/min/1.73 m² for patients with baseline eGFR≥30 mL/min/1.73 m², and sustained eGFR (CKD-EPI)<10 mL/min/1.73 m² for patients with baseline eGFR<30 mL/min/1.73 m², or need for chronic dialysis treatment or need for receiving a renal transplant-time to cardiovascular death
time to all-cause mortality
health related quality of life (for example as measured by KCCQ or KCCQ-12)
time to new onset of type 2 diabetes mellitus in non-diabetic patients
time to re-hospitalisation for heart failure
change in NYHA classification
time to all-cause hospitalisation, including first and/or recurrent,
time to new onset of atrial fibrillation
time to non-fatal or fatal myocardial infarction
time to non-fatal or fatal stroke
time to composite of cardiovascular death or myocardial infarction
time to composite of cardiovascular death or stroke
time to any of cardiovascular death (including fatal stroke, fatal myocardial infarction and sudden death), non-fatal myocardial infarction, non-fatal stroke (the so-called 3-point MACE)
change in eGFR
progression to macroalbuminuria (defined as albumin/creatinine ratio (ACR)≥300 mg/g)
time to need for chronic dialysis treatment
time to need for receiving a renal transplant
composite of eGFR reduction, renal replacement therapy or renal death
composite of eGFR reduction, renal replacement therapy, renal death or cardiovascular death
composite of eGFR reduction, renal replacement therapy, renal death or all-cause mortality.

### Example 2

### Treatment of Patients with Chronic Heart Failure and HFpEF

The longterm impact on cardiovascular death or hospitalization for heart failure and other parameters of treatment with empagliflozin in a relevant population of patients with chronic heart failure and preserved ejection fraction is investigated as follows:

Patients with chronic heart failure and symptoms according to NYHA II, III or IV and a preserved ejection fraction (LVEF greater than 40% or greater than 50%) are treated over a long term (e.g. for between approximately 20 to 38 months for each patient) with empagliflozin (optionally in combination with one or more other active substances, e.g. such as those described herein) and compared with patients who have been treated with a placebo on standard of care background medication.

Empagliflozin is administered orally once daily (for example 10 mg/daily). Patients include non-diabetic patients, patients with pre-diabetes and patients with type 2 diabetes mellitus.Pre-diabetes is diagnosed if HbA1c is more or equal to 5.7% and less than 6.5%. An individual is a non-diabetic patient if the HbA1c is less than 5.7%. Patients have a LVEF greater than 40%, in particular greater than 50%.

Patients include individuals who were hospitalized for heart failure within the last 9 months and/or have an elevated BNP≥75 pg/mL or NT-proBNP≥300 pg/mL (for patients not with atrial fibrillation (AF)) or an elevated BNP>225 pg/mL or NT-proBNP>900 pg/mL (for patients with atrial fibrillation (AF)).

The study is event-driven and all randomised patients will remain in the trial until the defined number of patients with primary endpoint events has been reached. The number of confirmed adjudicated primary endpoint events will be continuously monitored during the study.

The patients with cardiovascular risk factors are treated according to standard of care, which includes symptomatic treatment, and treatment of cardiovascular risk factors including hypertension, diabetes mellitus, and dyslipidemia.

Patients in the study follow the following criteria:
age above 18 years
Diagnosis of heart failure (HF). Definition of HF for inclusion in the study is left ventricular ejection fraction (LVEF) (ideally obtained by echocardiography, although radionuclide ventriculography and angiography are acceptable)>40% (per local reading). The ejection fraction value is preferably obtained within 6 months prior to randomization and after any Myocardial Infarction (MI) or other event that would affect the ejection fraction.
Symptom(s) of heart failure (HF) (NYHA class II-IV)
Structural heart disease (left atrial enlargement or left ventricular hypertrophy) documented by echocardiogram
At least one of the following: A heart failure hospitalization within the last 9 months and/or an elevated NT-proBNP (>300 pg/mL for patients not with atrial fibrillation (AF) or >900 pg/mL for patients with atrial fibrillation (AF)).
Background therapy for heart failure if needed
Antidiabetic background if needed
Body Mass Index (BMI)<45 kg/m²
eGFR≥20 mL/min/1.73 m² or eGFR≥30 mL/min/1.73 m²

The time to cardiovascular death or hospitalisation for heart failure is determined in patients with heart failure with preserved ejection fraction (according to the criteria as described hereinbefore) treated with empagliflozin (e.g. 10 mg once daily) to placebo.

One or more of the following events are determined:
time to first hospitalisation for heart failure
eGFR (CKD-EPI) slope of change from baseline
Time to first occurrence of sustained reduction of ≥40% eGFR (CKD-EPI)
Time to first occurrence of sustained eGFR (CKD-EPI)<15 mL/min/1.73 m² for patients with baseline eGFR≥30 mL/min/1.73 m²
Time to first occurrence of sustained eGFR (CKD-EPI)<10 mL/min/1.73 m² for patients with baseline eGFR<30 mL/min/1.73 m²
Composite of time to first occurence of sustained reduction of ≥40% eGFR (CKD-EPI) or sustained eGFR (CKD-EPI)<15 mL/min/1.73 m² for patients with baseline eGFR≥30 mL/min/1.73 m², sustained eGFR (CKD-EPI)<10 mL/min/1.73 m² for patients with baseline eGFR<30 mL/min/1.73 m²,
Composite of time to first occurence of sustained reduction of ≥40% eGFR (CKD-EPI) or sustained eGFR (CKD-EPI)<15 mL/min/1.73 m² for patients with baseline eGFR≥30 mL/min/1.73 m², sustained eGFR (CKD-EPI)<10 mL/min/1.73 m² for patients with baseline eGFR<30 mL/min/1.73 m², or need for chronic dialysis treatment or need for receiving a renal transplant
time to cardiovascular death
time to all-cause mortality
health related quality of life (for example as measured by KCCQ or KCCQ-12)
time to new onset of type 2 diabetes mellitus in non-diabetic patients
time to re-hospitalisation for heart failure
change in NYHA classification
time to all-cause hospitalisation, including first and/or recurrent,
time to new onset of atrial fibrillation
time to non-fatal or fatal myocardial infarction
time to non-fatal or fatal stroke
time to any of cardiovascular death (including fatal stroke, fatal myocardial infarction and sudden death), non-fatal myocardial infarction (excluding silent myocardial infarction), non-fatal stroke (the so-called 3-point MACE)
change in eGFR
progression to macroalbuminuria (defined as albumin/creatinine ratio (ACR)≥300 mg/g)
time to need for chronic dialysis treatment
time to need for receiving a renal transplant
composite of eGFR reduction, renal replacement therapy or renal death
composite of eGFR reduction, renal replacement therapy, renal death or cardiovascular death
composite of eGFR reduction, renal replacement therapy, renal death or all-cause mortality

The health related quality of life may be measured according to KCCQ or KCCQ-12. KCCQ-12 is a validated short version of the original 23-item KCCQ (Kansas City Cardiomyopathy Questionnaire). This self-administered questionnaire is designed to evaluate physical limitations, symptoms (frequency, severity, and changes over time), social limitations, self-efficacy, and quality of life in patients with HF.

### Example 3

### Treatment of Frail Patients with Chronic Heart Failure and HFrEF

The impact of a treatment with empagliflozin on the functional capacity and other parameters in a relevant population of patients with chronic heart failure and reduced ejection fraction and frailty is investigated as follows:

Patients with chronic heart failure and symptoms according to NYHA II, III or IV and a reduced ejection fraction (LVEF smaller or equal than 40%) and an elevated BNP (or elevated NT-proBNP), e.g. as defined below, and with frailty are treated over a period of time (e.g. for approximately 12 weeks for each patient) with empagliflozin (optionally in combination with one or more other active substances, e.g. such as those described herein) and compared with patients who have been treated with a placebo on standard of care background medication.

Empagliflozin is administered orally once daily (for example 10 mg/daily). Patients include non-diabetic patients, patients with pre-diabetes and patients with type 2 diabetes mellitus. Pre-diabetes is diagnosed if HbA1c is more or equal to 5.7% and less than 6.5%. An individual is a non-diabetic patient if the HbA1c is less than 5.7%.

Patients have a LVEF smaller or equal than 40%.

Patients with an elevated BNP (or elevated NT-proBNP) are defined as having one of the following:
an elevated BNP≥150 pg/mL or NT-proBNP≥600 pg/mL; or
if patients were hospitalized for heart failure within the last 9 months, an elevated BNP≥100 pg/mL or NT-proBNP≥400 pg/mL.

Patients with reduced ejection fraction may be included according to at least one of the following evidence of heart failure:

If the ejection fraction EF is ≥36% to ≤40% then the elevated NT-proBNP shall be ≥2500 pg/ml for patients without atrial fibrillation, or ≥5000 pg/ml for patients with atrial fibrillation.

If the ejection fraction EF is ≥31% to ≤35% then the elevated NT-proBNP shall be ≥1000 pg/ml for patients without atrial fibrillation, or ≥2000 pg/ml for patients with atrial fibrillation.

If the ejection fraction EF is ≤30% then the elevated NT-proBNP shall be ≥600 pg/ml for patients without atrial fibrillation, or ≥1200 pg/ml for patients with atrial fibrillation.

A patient with frailty is included in the study, if for example in 6 minutes walking test the patient manages a distance of less than 350 meters.

At the end of the study period for each patient the functional capacity, in particular the exercise capacity, for example a 6 minutes walking test, and further clinical parameters, for example as below, are investigated.

The patients with cardiovascular risk factors are treated according to standard of care, which includes for example treatment with therapeutic agents selected from diuretics, ARNi, ACEi, ARB, statins, aspirin, beta-blockers, mineral corticoid antagonist or ivabradine,with or without cardiac device therapy including ICD, CRT-D or CRT-P.

Patients in the study follow the following criteria:
age above 18 years
Diagnosis of heart failure (HF). Definition of HF for inclusion in the study is left ventricular ejection fraction (LVEF) (ideally obtained by echocardiography, although radionuclide ventriculography and angiography are acceptable) smaller or equal than 40% (per local reading). The ejection fraction value is preferably obtained within 6 months prior to randomization and after any Myocardial Infarction (MI) or other event that would affect the ejection fraction.
Symptom(s) of heart failure (HF) (NYHA class II-IV)
At least one of the following: an elevated NT-proBNP≥600 pg/mL and/or if the patient was hospitalized for heart failure within the last 9 months an elevated NT-proBNP≥400 pg/mL.
frailty, for example determined via a 6 minutes walking test in which the patient manages a distance of less than 350 meters.
Background therapy for heart failure if needed
Antidiabetic background if needed
Body Mass Index (BMI)<45 kg/m²
eGFR≥20 mL/min/1.73 m² or eGFR≥30 mL/min/1.73 m²

At the end of a defined period of time, e.g. 12 weeks, the functional capacity, in particular the exercise capacity, for example a 6 minutes walking test, is determined in the patients with heart failure with reduced ejection fraction (according to the criteria as described hereinbefore) treated with empagliflozin (e.g. 10 mg once daily) or placebo.

One or more of the following events are determined:
change in NYHA classification
health related quality of life (for example as measured by KCCQ or KCCQ-12, MLHFQ, fatigue score, depression score, anxiety score, global assessment score)
change from baseline biomarkes, e.g. NT-proBNP
time to first hospitalisation for heart failure
time to re-hospitalisation for heart failure.

### Example 4

### Treatment of Frail Patients with Chronic Heart Failure and HFpEF

The impact of a treatment with empagliflozin on the functional capacity and other parameters in a relevant population of patients with chronic heart failure and preserved ejection fraction and frailty is investigated as follows:

Patients with chronic heart failure and symptoms according to NYHA II, III or IV and a preserved ejection fraction (LVEF greater than 40% or greater than 50%) and with frailty are treated over a period of time (e.g. for approximately 12 weeks for each patient) with empagliflozin (optionally in combination with one or more other active substances, e.g. such as those described herein) and compared with patients who have been treated with a placebo on standard of care background medication.

Empagliflozin is administered orally once daily (for example 10 mg/daily). Patients include non-diabetic patients, patients with pre-diabetes and patients with type 2 diabetes mellitus. Pre-diabetes is diagnosed if HbA1c is more or equal to 5.7% and less than 6.5%. An individual is a non-diabetic patient if the HbA1c is less than 5.7%.

Patients have a LVEF greater than 40%, in particular greater than 50%.

Patients include individuals who were hospitalized for heart failure within the last 9 months and/or have an elevated BNP≥75 pg/mL or NT-proBNP≥300 pg/mL (for patients not with atrial fibrillation (AF)) or an elevated BNP>225 pg/mL or NT-proBNP>900 pg/mL (for patients with atrial fibrillation (AF)).

A patient with frailty is included in the study, if for example in 6 minutes walking test the patient manages a distance of less than 350 meters.

At the end of the study period for each patient the functional capacity, in particular the exercise capacity, for example a 6 minutes walking test, and further clinical parameters, for example as below, are investigated.

The patients with cardiovascular risk factors are treated according to standard of care, which includes symptomatic treatment, and treatment of cardiovascular risk factors including hypertension, diabetes mellitus, and dyslipidemia.

Patients in the study follow the following criteria:
age above 18 years
Diagnosis of heart failure (HF). Definition of HF for inclusion in the study is left ventricular ejection fraction (LVEF) (ideally obtained by echocardiography, although radionuclide ventriculography and angiography are acceptable)>40% (per local reading). The ejection fraction value is preferably obtained within 6 months prior to randomization and after any Myocardial Infarction (MI) or other event that would affect the ejection fraction.
Symptom(s) of heart failure (HF) (NYHA class II-IV)
Structural heart disease (left atrial enlargement or left ventricular hypertrophy) documented by echocardiogram
At least one of the following: A heart failure hospitalization within the last 9 months and/or an elevated NT-proBNP (>300 pg/mL for patients not with atrial fibrillation (AF) or >900 pg/mL for patients with atrial fibrillation (AF)).
Frailty, for example determined via a 6 minutes walking test in which the patient manages a distance of less than 350 meters.
Background therapy for heart failure if needed
Antidiabetic background if needed
Body Mass Index (BMI)<45 kg/m²
eGFR≥20 mL/min/1.73 m² or eGFR≥30 mL/min/1.73 m²

At the end of a defined period of time, e.g. 12 weeks, the functional capacity, in particular the exercise capacity, for example a 6 minutes walking test, is determined in the patients with heart failure with preserved ejection fraction (according to the criteria as described hereinbefore) treated with empagliflozin (e.g. 10 mg once daily) or placebo.

One or more of the following events are determined:
change in NYHA classification
health related quality of life (for example as measured by KCCQ or KCCQ-12, MLHFQ, fatigue score, depression score, anxiety score, global assessment score)
change from baseline biomarkes, e.g. NT-proBNP
time to first hospitalisation for heart failure
time to re-hospitalisation for heart failure.

Example of Pharmaceutical Composition and Dosage Form

The following example of solid pharmaceutical compositions and dosage forms for oral administration serves to illustrate the present invention more fully without restricting it to the contents of the example. Further examples of compositions and dosage forms for oral administration, are described in WO 2010/092126. The term “active substance” denotes empagliflozin according to this invention, especially its crystalline form as described in WO 2006/117359 and WO 2011/039107.

Tablets containing 2.5 mg, 5 mg, 10 mg or 25 mg of the active substance empagliflozin. Amounts of the ingredients are provided in mg per film-coated tablet.

| | 2.5 mg/ | 5 mg/ | 10 mg/ | 25 mg/ |
|---|---|---|---|---|
| Active substance | per tablet | per tablet | per tablet | per tablet |
| Wet granulation | | | | |
| empagliflozin | 2.5000 | 5.000 | 10.00 | 25.00 |
| Lactose | 40.6250 | 81.250 | 162.50 | 113.00 |
| Monohydrate | | | | |
| Microcrystalline | 12.5000 | 25.000 | 50.00 | 40.00 |
| Cellulose | | | | |
| Hydroxypropyl | 1.8750 | 3.750 | 7.50 | 6.00 |
| Cellulose | | | | |
| Croscarmellose | 1.2500 | 2.500 | 5.00 | 4.00 |
| Sodium | | | | |
| Purified Water | q.s. | q.s. | q.s. | q.s. |
| Dry Adds | | | | |
| Microcrystalline | 3.1250 | 6.250 | 12.50 | 10.00 |
| Cellulose | | | | |
| Colloidal silicon | 0.3125 | 0.625 | 1.25 | 1.00 |
| dioxide | | | | |
| Magnesium stearate | 0.3125 | 0.625 | 1.25 | 1.00 |
| Total core | 62.5000 | 125.000 | 250.00 | 200.00 |
| Film Coating | | | | |
| Film coating system | 2.5000 | 4.000 | 7.00 | 6.00 |
| Purified Water | q.s. | q.s. | q.s. | q.s. |
| Total | 65.000 | 129.000 | 257.00 | 206.00 |

Details regarding the manufacture of the tablets, the active pharmaceutical ingredient, the excipients and the film coating system are described in WO 2010/092126, in particular in the Examples 5 and 6, which hereby is incorporated herein in its entirety.

1. A method for treating, preventing, protecting against or delaying the occurrence of chronic heart failure in a patient in need thereof comprising administering empagliflozin to the patient.

2. A method for:
reducing the risk of cardiovascular death;
reducing the risk of hospitalization for heart failure;
reducing all-cause mortality;
reducing the risk of all-cause hospitalization; or
reducing the risk of new onset of atrial fibrillation in a patient with chronic heart failure comprising administering empagliflozin to the patient.

3. The method according to claim 2, wherein the risk of hospitalization for heart failure is the risk of first hospitalization for heart failure.

4. A method for treating, preventing, protecting against, reducing the risk of or delaying the occurrence of acute heart failure, including acute decompensated heart failure, in a patient in need thereof comprising administering empagliflozin to the patient.

5. A method for treating, preventing, protecting against, reducing the risk of, delaying the occurrence of and/or delaying the progression of chronic kidney disease in a patient with chronic heart failure comprising administering empagliflozin to the patient.

6. A method for improving the health related quality of life and/or the functional capacity in a patient with chronic heart failure comprising administering empagliflozin to the patient.

7. The method according to claim 1, wherein the patient is a patient with chronic heart failure according to NYHA class II, III or IV.

8. The method according to claim 1, wherein the patient is a patient with preserved ejection fraction.

9. The method according to claim 1, wherein the patient is a patient with reduced ejection fraction.

10. The method according to claim 1, wherein the patient is a patient with pre-diabetes, type 1 diabetes mellitus or type 2 diabetes mellitus.

11. The method according to claim 1, wherein the patient is a non-diabetic patient.

12. The method according to claim 1, wherein the patient has an eGFR equal to or greater than 20 mL/min/1.73 m² or eGFR equal to or greater than 30 mL/min/1.73 m² or eGFR equal to or greater than 45 mL/min/1.73 m² or eGFR equal to or greater than 60 mL/min/1.73 m².

13. The method according to claim 1, wherein empagliflozin is administered at a dose in a range from 1 mg to 25 mg.

14. The method according to claim 1, wherein empagliflozin is administered in combination with one or more other therapeutic substances to the patient.

15. The method according to claim 14, wherein the one or more other therapeutic substances are selected from the group consisting of active substances that are indicated in the treatment of chronic heart failure, antidiabetic substances, active substances that lower the total cholesterol, LDL-cholesterol, Non-HDL-cholesterol and/or Lp(a) level in the blood, active substances that raise the HDL-cholesterol level in the blood, active substances that lower blood pressure, active substances that are indicated in the treatment of atherosclerosis or obesity, antiplatelet agents, anticoagulant agents, and vascular endothelial protective agents.
